(19) United States
(12) Patent Application Publication
(10) Pub. No.: US 2008/0138384 A1
(43) Pub. Date: Jun. 12, 2008

(54) ORAL HAIR GROWTH STIMULANTS COMPRISING ORTHOSIPHON

(76) Inventors: Kouichi Nakaoji, Osaka (JP); Masahiro Matsuura, Osaka (JP); Naoko Hayashi, Osaka (JP); Toyoyuki Nishi, Kyoto-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 12/029,709

(22) Filed: Feb. 12, 2008

Related U.S. Application Data
(62) Division of application No. 10/525,655, filed on Feb. 25, 2005.

(30) Foreign Application Priority Data
Aug. 28, 2002 (JP) 2002-248579

(51) Int. Cl. A61K 36/53; A61K 9/28; A61K 9/68; A61Q 7/00

(52) U.S. Cl. 424/440; 424/745; 424/441

(57) ABSTRACT

Disclosed is a hair growth stimulant for oral use comprising at least one member selected from the group consisting of plants of the genus Orthosiphon in the family Lamiaceae, plants of the genus Vitex L. in the family Verbenaceae, and extracts thereof; and a food comprising the hair growth stimulant for oral use.

### CROSS-REFERENCE TO PRIORITY AND RELATED APPLICATIONS

This application is a divisional application of U.S. patent application Ser. No. 10/525,655, filed Feb. 25, 2005, which is a U.S. National Phase Filing under 35 U.S.C. § 371 of the PCT Application No. PCT/JP2003/010820, filed Aug. 27, 2003, which claims priority to Japanese Patent Application No. 2002-248579, filed Aug. 28, 2002. The disclosures of each of the above-referenced applications are incorporated by reference herein.

### TECHNICAL FIELD

The present invention relates to a hair growth stimulant for oral use that exhibits excellent effects for hair loss prevention, hair regrowth, etc.

### BACKGROUND ART

Recently, an increasing number of men and women are said to be suffering from thinning hair or hair loss caused by various changes in their social environment, such as an increase in stress factors and changes in eating habits, and thus the social expectations and needs for hair restorers have been increasing. Hitherto, hair restorers containing a variety of active ingredients that are intended to eliminate or alleviate the causes of thinning hair or hair loss have been developed. For example, hair restorers containing swertia herb extract or tocopherol acetate to increase the blood flow to the hair roots, or hinokitiol to improve scalp metabolism have been developed and used to prevent or treat alopecia.

It is believed, however, that various factors, such as hereditary factors, stress, eating habits and aging, are combined in a complicated manner to cause thinning hair or hair loss. Thus, no satisfactory effects of hair loss prevention or hair regrowth can be obtained simply by adding swertia herb extract or other ingredients to improve blood flow or scalp metabolism, like hitherto known hair restorers.

Cat's whiskers (Orthosiphon aristatus (Blume) Miq.) and Orthosiphon stamineus Benth. are perennial plants of the genus Orthosiphon in the family Lamiaceae, and distributed across India, Southeast Asia, the northern part of Australia, Okinawa in Japan, and elsewhere. In India, Malaysia and Indonesia, these plants are used as important folk medicines and known to have effects for alleviating the symptoms of acute or chronic nephritis, cystitis, urolithiasis, cholelithiasis, rheumatic arthritis, diabetes and other diseases. Especially in Indonesia, these plants are called “kumis kuching” and are taken as effective folk medicines against nephritis, urolithiasis, etc.

Japanese Unexamined Patent Publication Nos. 1998-29924, 1999-228337, 2000-95663 and 2001-224330 reported use of plants of the genus Orthosiphon in the family Lamiaceae or extracts thereof as external medicines to be applied to the scalp, such as hair tonics or hair restorers. However, these medicines do not have satisfactory effects.

Further, lilac chastetree (Vitex agnus-castus L.), simpleleaf chastetree (Vitex trifolia L.) and other plants are known as plants of the genus Vitex L. in the family Verbenaceae.

Lilac chastetree is distributed from Southern Europe to the western part of Asia. In Europe, its fruit is used as a flavoring or perfume and its leaves and fruit as medicines, such as a diuretic, a cold medicine, an aphrodisiac for males, an antidepressant for menopause, etc. DE3442961 reported use of the plant body or an extract of lilac chastetree as a hair growth stimulant for external use, but with unsatisfactory effects.

Simpleleaf chastetree is widely distributed from the Korean Peninsula to Southeast Asia and Australia. In China, the fruit of simpleleaf chastetree is called Man Jing Zi, and is used for the treatment of headaches and colds, alleviation of fever, pain relief, sedation, anti-inflammatory treatment, etc. The Chuyaku Jiten (Dictionary of Chinese Medicines; pp. 2456-2458) and Japanese Unexamined Patent Publication Nos. 1984-116211 and 2000-31512 reported use of the plant body or an extract of simpleleaf chastetree as an external medicine to be applied to the scalp. However, the external medicine, such as a hair tonic or hair restorer, does not exhibit satisfactory effects.

Use of plants of the genus Orthosiphon in the family Lamiaceae or the genus Vitex L. in the family Verbenaceae as a hair growth stimulant for oral use has not hitherto been known.

### DISCLOSURE OF THE INVENTION

The main object of the present invention is to provide a hair growth stimulant for oral use that has excellent hair growth activity and significant effects for hair loss prevention, hair regrowth promotion, etc.

The present inventors conducted extensive research to achieve the above object, and found that oral administration of a plant of the genus Orthosiphon in the family Lamiaceae or the genus Vitex L. in the family Verbenaceae or an extract of the plant exhibits a higher hair growth effect than external use thereof. The inventors carried out further research and accomplished the present invention.

The present invention provides the following hair growth stimulants for oral use and food for hair growth.

1. A hair growth stimulant for oral use comprising at least one member selected from the group consisting of plants of the genus Orthosiphon in the family Lamiaceae, plants of the genus Vitex L. in the family Verbenaceae, and extracts thereof.

2. The hair growth stimulant for oral use according to item 1, wherein the plants of the genus Orthosiphon in the family Lamiaceae are at least one member selected from the group consisting of cat's whiskers (Orthosiphon Aristatus (Blume) Miq.)), Orthosiphon grandiflorus Bold., Orthosiphon rubicundus Benth., Orthosiphon spicatus Benth., and Orthosiphon stamineus Benth.

3. The hair growth stimulant for oral use according to item 1, wherein the plants of the genus Vitex L. in the family Verbenaceae are at least one member selected from the group consisting of simpleleaf chastetree (Vitex trifolia L.), roundleaf chastetree (Vitex rotundifolia L.f.), hempleaf negundo chastetree (Vitex cannabifolia Sieb.et Zucc.), negundo chastetree (Vitex negundo L.), and lilac chastetree (Vitex agnus-castus L.).

4. The hair growth stimulant for oral use according to any one of items 1 to 3, which is a food for hair growth.

The present invention is described below in detail.

### Plants of the Genus Orthosiphon in the Family Lamiaceae

Examples of plants of the genus Orthosiphon in the family Lamiaceae usable in the present invention include cat's whiskers (Orthosiphon aristatus (Blume) Miq.)), Orthosiphon grandiflorus Bold., Orthosiphon rubicundus Benth., Orthosiphon spicatus Benth., Orthosiphon stamineus Benth., etc.

In the present invention, plants of the genus Orthosiphon in the family Lamiaceae may be used as ground products of raw or dried plants, or as extracts obtained by effectively extracting active ingredients from the plants.

The part of the plants to be used is not limited. The aboveground parts, such as stems, leaves and flowers, the subterranean parts, such as roots, or the whole plant can be used, with the aboveground parts being especially preferable.

Extracts of the plants can be obtained by chopping the raw or dried plant and subjecting the chopped plant to extraction with a suitable solvent, which is preferably water, a lower alcohol or a mixture thereof.

Lower alcohols usable for extraction include C₁₋₃ alcohols, such as methanol, ethanol, etc., among which ethanol is most preferable. These alcohols may also be hydrous alcohol (water content: 0.1 to 99.9%).

The extraction can be performed by a batch process, percolation process, reflux process or other known processes. The proportion of the extraction solvent is not limited, and is suitably 2 to 1000 parts relative to 1 part of the chopped raw or dried plant. The extraction can be carried out at room temperature or with heating.

Preferably, the extraction is performed at a temperature range of room temperature to about 80° C. for about 1 hour to about 10 hours while stirring under mild conditions. The extraction may also be carried out by adding the solvent dropwise to a cylinder filled with the chopped raw or dried plant.

### Plants of the Genus Vitex L. in the Family Verbenaceae

Examples of plants of the genus Vitex L. in the family Verbenaceae usable in the present invention include simpleleaf chastetree (Vitex trifolia L.), roundleaf chastetree (Vitex rotundifolia L.f.), hempleaf negundo chastetree (Vitex cannabifolia Sieb.et Zucc.), negundo chastetree (Vitex negundo L.), and lilac chastetree (Vitex agnus-castus L.).

Preferable examples include lilac chastetree and simpleleaf chastetree.

In the present invention, plants of the genus Vitex L. in the family Verbenaceae can be used as ground products of raw or dried plants, or as extracts obtained by effectively extracting active ingredients from the plants.

The part of the plants to be used is not limited, and the roots, trunks, branches, leaves, flowers, fruit and other parts are usable.

Extracts of the plants can be obtained by chopping the raw or dried plant and then subjecting the chopped plant to extraction with a suitable solvent, which is preferably water, a lower alcohol or a mixture thereof.

Lower alcohols usable for extraction include C₁₃ alcohols, such as methanol, ethanol, etc., among which ethanol is most preferable. The alcohols may be hydrous alcohol (water content: 0.1 to 99.9%).

The extraction can be performed by a batch process, percolation process, reflux process or other known processes. The amount of the extraction solvent is not limited, and is suitably 2 to 1000 parts relative to 1 part of the chopped raw or dried plant. The extraction can be carried out at room temperature or with heating.

Preferably, the extraction is performed at a temperature range of room temperature to about 80° C. for about 1 hour to about 10 hours while stirring under mild conditions. The extraction may be carried out by adding the solvent dropwise to a cylinder filled with the chopped raw or dried plant.

### Hair Growth Stimulant for Oral Use

The hair growth stimulant for oral use of the present invention comprises, as an active ingredient, at least one member selected from the group consisting of plants of the genus Orthosiphon in the family Lamiaceae, plants of the genus Vitex L. in the family Verbenaceae, and extracts thereof.

The hair growth stimulant for oral use of the present invention can be used as a preparation that is suitable as a health food, functional food, supplement or the like. The preparation can also be used as a medicine.

The hair growth stimulant for oral use of the present invention may consist solely of a plant or plants of the genus Orthosiphon in the family Lamiaceae, a plant or plants of the genus Vitex L. in the family Verbenaceae and/or an extract or extracts thereof, or may be prepared by mixing the plant(s) and/or extract(s) with a material that is used as a carrier and then processing the mixture into a powder, mass, liquid or other form.

Since plants of the genus Orthosiphon in the family Lamiaceae and the genus Vitex L. in the family Verbenaceae, and extracts thereof, show no toxicity for humans, the amount of the plant or plants and/or an extract or extracts thereof contained in the hair growth stimulant for oral use is not limited, and is preferably such that an adult human can orally ingest 0.1 to 100 g, and preferably 1 to 60 g (calculated as dried plant) of the plant or plants and/or extract or extracts thereof per day, divided into 1 to 3 doses.

The hair growth stimulant for oral use of the present invention may contain suitable additives as required.

The hair growth stimulant for oral use of the present invention can be formulated into an easily usable form, such as tablets, powder, granules, capsules, chewable tablets, liquid, etc.

### Food

The hair growth stimulant for oral use of the present invention can be used in a so-called “food form”, such as a food for hair growth.

The food comprising the hair growth stimulant for oral use of the present invention, such as a food for hair growth, can be prepared by processing the hair growth stimulant for oral use alone, or by processing a mixture of the hair growth stimulant for oral use and an arbitrary material usable for food preparation, into a powder, mass, liquid or other form.

The food comprising the hair growth stimulant for oral use of the present invention used for a food can be made into various forms, such as a mass, liquid, syrup, powder, jelly, etc., in a routine manner.

Examples of food forms include soft drinks, juices, teas and other beverages (drink preparations); powdered juices, powdered soups and other powdered food; cookies, biscuits, cereals, chewing gums, candies, gummy candies, tablets, wafers, rice crackers and other confections and snacks; etc.

The food comprising the hair growth stimulant for oral use of the present invention may further contain other ingredients generally used in foods, to such an extent that the effects of the invention are not hindered. Examples of usable ingredients include other medicinal ingredients, nutrients, animal and plant ingredients, excipients, extenders, sweeteners, flavors, colors, preservatives, emulsifiers, solubilizers, polyhydric alcohols and ester derivatives thereof, organic and inorganic acids and salts thereof, water-soluble polymers, etc.

### BEST MODE FOR CARRYING OUT THE INVENTION

The following Experiment and Examples are intended to illustrate the present invention in further detail, and not to limit the scope thereof.

### Experiment

(i) Preparation of Extract

One hundred grams of a dried whole plant of cat's whiskers, simpleleaf chastetree or lilac chastetree was chopped. Ethanol (2000 ml) was added, and the resulting mixture was heated to 70° C. for 2 hours to perform extraction. After filtration, 2000 ml of ethanol was further added to the residue, and extraction was carried out for 2 hours more. The filtrates obtained by the two extraction steps were combined, concentrated under reduced pressure, and freeze-dried to obtain an extract.

(ii) Test for Hair Regrowth Promotion by External or Oral Use, and Evaluation of the Test Results

An area of about 2×4 cm was shaved on the back of nine 8-week-old C3H/He male mice (average weight: 25 g).

From the day following the day of shaving, 0.1 ml of a 50% aqueous ethanol solution containing 1 wt. % of the extract obtained in (i) was applied to the shaved area of the group for testing external use, once a day for 10 days.

In the group for testing oral use, a 0.5% aqueous carboxymethylcellulose solution containing 5 wt. % of the extract obtained in (i) was orally administered in a dose containing 1 g of the extract per kg of body weight, once a day for 10 days.

Hair regrowth effect was evaluated by comparing the hair restoration in the shaved area. The average of the hair restoration results for the nine mice in each group was used for the comparison.

The hair regrowth promotion effect was expressed as a percentage relative to the hair restoration of the control group (external use of 50% ethanol or oral use of 0.5% carboxymethylcellulose), which was set to 100%, and the increment was presented as a hair regrowth promotion ratio. Table 1 shows the test results of the cat's whiskers extract.

TABLE 1

| | | | Hair regrowth |
|---|---|---|---|
| | | Hair restoration | promotion ratio |
| | Test substance | ratio (%) | (%) |
| External use | Control | 100 | |
| | Cat's whiskers extract | 121 | 21 |
| Oral use | Control | 100 | |
| | Cat's whiskers extract | 185 | 85 |

Table 1 reveals that the oral use of the cat's whiskers extract exhibits a higher hair regrowth promotion effect than the external use thereof. Similar results were obtained with respect to the simpleleaf chastetree extract and lilac chastetree extract.

### EXAMPLE 1

### Formulation of Sugar-Coated Tablets

The extracts prepared in the Experiment and other ingredients were mixed in the proportions shown in Examples 1-A to 1-C, and made into sugar-coated tablets in a routine manner.

### EXAMPLE 1-A

| Ingredient | Proportion (%) |
|---|---|
| Dolomite | Balance |
| (containing 20% of calcium and | |
| 10% of magnesium) | |
| Powdered maltitol syrup | 20 |
| Lactose | 17 |
| Sucrose fatty acid ester | 3 |
| Cat's whiskers extract | 5 |

### EXAMPLE 1-B

| Ingredient | Proportion (%) |
|---|---|
| Dolomite | Balance |
| (containing 20% of calcium and | |
| 10% of magnesium) | |
| Powdered maltitol syrup | 20 |
| Lactose | 17 |
| Sucrose fatty acid ester | 3 |
| Lilac chastetree extract | 5 |

### EXAMPLE 1-C

| Ingredient | Proportion (%) |
|---|---|
| Dolomite | Balance |
| (containing 20% of calcium and | |
| 10% of magnesium) | |
| Powdered maltitol syrup | 20 |
| Lactose | 17 |
| Sucrose fatty acid ester | 3 |
| Simpleleaf chastetree extract | 5 |

### EXAMPLE 2

### Formulation of Tablets

The extracts prepared in the Experiment and other ingredients were mixed in the proportions shown in Examples 2-A to 2-C, and made into tablets in a routine manner.

### EXAMPLE 2-A

| Ingredient | Proportion (%) |
|---|---|
| Dextrin | Balance |
| Powdered maltitol syrup | 20 |
| Lactose | 20 |
| Trehalose | 10 |
| Cat's whiskers extract | 5 |
| Aspartame | Trace |
| Flavor | Trace |

### EXAMPLE 2-B

| Ingredient | Proportion (%) |
|---|---|
| Dextrin | Balance |
| Powdered maltitol syrup | 20 |
| Lactose | 20 |
| Trehalose | 10 |
| Lilac chastetree extract | 5 |
| Aspartame | Trace |
| Flavor | Trace |

### EXAMPLE 2-C

| Ingredient | Amount (%) |
|---|---|
| Dextrin | Balance |
| Powdered maltitol syrup | 20 |
| Lactose | 20 |
| Trehalose | 10 |
| Simpleleaf chastetree extract | 5 |
| Aspartame | Trace |
| Flavor | Trace |

### EXAMPLE 3

### Formulation of Drink Preparation

The extracts prepared in the Experiment and other ingredients were mixed in the proportions shown in Examples 3-A to 3-C, and made into drink preparations in a routine manner.

### EXAMPLE 3-A

| Ingredient | Amount (%) |
|---|---|
| Cat's whiskers extract | 5 |
| Sucrose | 2 |
| Ascorbic acid | 1.5 |
| Preservative | Trace |
| Flavor | Trace |
| Purified Water | Balance |

### EXAMPLE 3-B

| Ingredient | Proportion (%) |
|---|---|
| Lilac chastetree extract | 5 |
| Sucrose | 2 |
| Ascorbic acid | 1.5 |
| Preservative | Trace |
| Flavor | Trace |
| Purified water | Balance |

### EXAMPLE 3-C

| Ingredient | Amount (%) |
|---|---|
| Simpleleaf chastetree extract | 5 |
| Sucrose | 2 |
| Ascorbic acid | 1.5 |
| Preservative | Trace |
| Flavor | Trace |

Purified water Balance

### INDUSTRIAL APPLICABILITY

The hair growth stimulant for oral use of the present invention, i.e., a hair growth stimulant for oral use comprising at least one member selected from the group consisting of plants of the genus Orthosiphon in the family Lamiaceae, plants of the genus Vitex L. in the family Verbenaceae, and extracts thereof, has an excellent hair growth activity and exhibits excellent effects of hair loss prevention, hair regrowth promotion, etc. Further, the food comprising the hair growth stimulant shows excellent effects of hair loss prevention, hair regrowth promotion, etc., and are useful as foods for hair regrowth and like purposes.

1. A method of stimulating hair growth comprising administrating orally to a subject desiring hair growth stimulation an effective amount of at least one member selected from the group consisting of plants of the genus Orthosiphon in the family Lamiaceae, and extracts thereof.

2. The method according to claim 1, wherein the plants of the genus Orthosiphon in the family Lamiaceae are cat's whiskers (Orthosiphon aristatus (Blume) Miq., Orthosiphon spicatus Benth., and Orthosiphon stamineus Benth.), Orthosiphon grandiflorus Bold., and Orthosiphon rubicundus Benth.

3. The method according to claim 1, wherein the at least one member is an aqueous extract of cat's whiskers (Orthosiphon aristatus (Blume) Miq., Orthosiphon spicatus Benth., and Orthosiphon stamineus Benth.).

4. The method according to claim 1, wherein the oral administration is conducted by providing an aqueous extract of the aboveground parts of the selected plant.

5. The method according to claim 1, wherein the effective amount is 1 to 60 g calculated as dried plant per day.

6. The method according to claim 1, wherein the oral administration is conducted daily and repeated for at least 10 days.

7. The method according to claim 1, wherein the oral administration is conducted by providing the at least one member in the form of food.

8. The method according to claim 1, wherein the oral administration is conducted by providing an extract of the selected plant, which extract is obtained using a solvent selected from the group consisting of water, C1-3 alcohol, and a mixture thereof.

9. The method according to claim 1, wherein the oral administration is conducted by providing at least one form selected from the group consisting of soft drinks, juices, teas, powdered juices, powdered soups, cookies, biscuits, cereals, chewing gums, candies, gummy candies, tablets, wafers, rice crackers and snacks.

10. The method according to claim 1, wherein the oral administration is conducted by providing at least one form selected from the group consisting of tablets, powder, granules, capsules and chewable tablets.

11. The method according to claim 1, wherein the oral administration is conducted by providing at least one form selected from the group consisting of a mass, syrup, powder and jelly.

12. The method according to claim 1, wherein the oral administration is conducted by providing the at least one member in the form of a liquid.
